(19) United States
(12) Patent Application Publication
(10) Pub. No.: US 2018/0243251 A1
(43) Pub. Date: Aug. 30, 2018

(54) Composition and Method for Improving Cognitive Function

(71) Applicant: Ioffe Biotechnologies Inc., Kingston (CA)

(72) Inventor: Altair Ioffe, Kingston (CA)

(21) Appl. No.: 15/903,575

(22) Filed: Feb. 23, 2018

Related U.S. Application Data
(60) Provisional application No. 62/463,239, filed on Feb. 24, 2017.

(51) Int. Cl. A61K 31/198; A61K 31/522; A61K 31/4415; A61K 31/405; A61K 36/41; A61P 25/28

(52) U.S. Cl. CPC A61K 31/198; A61K 31/522; A61P 25/28; A61K 31/405; A61K 36/41; A61K 31/4415

(57) ABSTRACT

Embodiments of the invention provide a composition for improving cognitive function in a subject. The composition includes caffeine, mood enhancers, and neutrotransmitter precursors.

### RELATED APPLICATION

This application claims the benefit of the filing date of U.S. Provisional Patent Application No. 62/463,239, filed on Feb. 24, 2017, the contents of which are incorporated herein by reference in their entirety.

### FIELD

The invention relates to compositions and methods for improving cognitive function.

### BACKGROUND

Caffeine is the most widely consumed psychoactive substance. This central nervous system (CNS) stimulant may be consumed to enhance wakefulness and cognitive performance, typically by ingesting beverages that include caffeine, such as coffee. A limitation of such a caffeine source is the lack of complementary ingredients to compensate for acute neurochemical imbalances and assist with reducing negative side-effects of caffeine consumption such as anxiety, jitteriness, and headache.

Traditional stimulants that improve cognitive performance act on neurotransmission mechanisms in the brain. One common mechanism includes the release of neurotransmitters involved in motivation, attention, and learning, such as dopamine, norepinephrine, and serotonin. Such traditional stimulants are not designed to replenish these neurotransmitters once they are released and degraded in the brain.

Currently available ingestible products that are marketed as boosting cognitive performance, such as Brainergy (EVO-X Health Products (USA)) similarly lack effective ingredients or sufficient amounts of effective ingredients to effectively replenish neurotransmitters and/or minimize side effects.

### SUMMARY

An aspect of the invention provides a composition comprising L-tyrosine, caffeine, L-theanine, Rhodiola rosea extract, vitamin B6, and a serotonin precursor. In one embodiment, vitamin B6 is pyridoxine, pyridoxamine, pyridoxal, or a salt thereof. In one embodiment, the vitamin B6 is pyridoxine hydrochloride (HCL). In one embodiment, the serotonin precursor is 5-hydroxytryptophan (5-HTP).

Another aspect of the invention provides a composition comprising L-tyrosine, caffeine, L-theanine, Rhodiola rosea extract, pyridoxine HCL, and 5-hydroxytryptophan (5-HTP). In some embodiments, the dosage amount of L-tyrosine is about 200 to about 1000 mg. In some embodiments, the dosage amount of caffeine is about 50 to about 250 mg. In some embodiments, the dosage amount of L-theanine is about 50 to about 400 mg. In some embodiments, the dosage amount of Rhodiola rosea extract is about 20 to about 200 mg. In some embodiments, the Rhodiola rosea extract comprises about 3% salidroside. In some embodiments, the dosage amount of pyridoxine HCl is about 10 to about 50 mg. In some embodiments, the dosage amount of 5-HTP is about 5 to about 50 mg. In some embodiments, the composition is orally-administered or transdermally-administered. In certain embodiments, the composition is in capsule form. In other embodiments, the composition is in powder form. In other embodiments, the composition is in liquid form. In other embodiments, the composition is a dietary supplement. In other embodiments, the composition is included in food or drink.

In one embodiment, the composition comprises 500 mg of L-tyrosine, 200 mg of caffeine, 160 mg of L-theanine, 80 mg of Rhodiola rosea extract, 40 mg of pyridoxine HCL, and 20 mg of 5-HTP.

In another aspect, the invention provides use of the composition of the above embodiments for improving cognitive performance.

Another aspect of the invention provides a method of improving cognitive performance comprising ingesting the composition of the above embodiments. In one embodiment of this aspect, the composition is ingested on an empty stomach (i.e., about an hour since food was consumed).

Another aspect of the invention provides a composition for enhancing cognitive function comprising L-tyrosine, caffeine, L-theanine, Rhodiola rosea extract, pyridoxine HCL, and 5-HTP.

### DETAILED DESCRIPTION OF EMBODIMENTS

Described herein is a cognitive stimulant composition that provides caffeine together with ingredients that alleviate caffeine's negative side effects as well as ingredients that provide the body with what it needs to replenish neurotransmitters. A cognitive stimulant composition as described herein includes one or more components that compensates for acute neurochemical imbalances. Such imbalances are typically induced by stimulants, such as caffeine. Such one or more compensating components thus supports biological demands of cognitive exertion, while attenuating side-effects of other components such as, for example, caffeine.

One aspect of the invention relates to a composition that stimulates the brain to improve cognitive performance, provides building blocks for the body to replenish neurotransmitters, and diminishes, reduces, or eliminates negative side effects of caffeine consumption. Examples of enhancing cognitive performance include increasing wakefulness and/or mental acuity. Examples of negative side effects include jitteriness, anxiety, headache, and other undesired effects that are associated with caffeine intake. Through a combination of ingredients, the composition described herein enhances cognitive performance and diminishes, reduces, or eliminates such negative side effects in a user. The term “user” refers to a subject that ingests the composition. The embodiments are intended primarily, but not exclusively, for use with human subjects. Thus, provided herein are compositions for consumption that may be used to provide desired effects of enhancing cognitive performance and replenishing neurotransmitters, and inhibiting undesired effects of caffeine.

As used herein, the term “dosage” refers to a recommended ingestion amount for a subject that is of average mass (e.g., 50-100 kg). For a subject whose body mass falls outside of the average range, ingestion amounts may be adjusted accordingly.

In some embodiments, the composition is provided in capsule form for oral consumption (i.e., ingestion). In other embodiments, the composition is provided in a powder form, and may be added to food or drink for ingestion. In some embodiments, the composition is formulated as a dietary supplement. In some embodiments, the supplement is provided in a liquid form for ingestion as drops, or in measured portions (e.g., mL, mg, or spoonfuls, etc.). In some embodiments, the composition may be applied transdermally. In some embodiments, the composition may be added to food for ingestion by eating. In some embodiments, the composition may be added to a liquid for ingestion by drinking.

The composition is a stimulant because one of its ingredients is caffeine. Caffeine increases wakefulness and cognitive function, but may cause side effects which are undesirable, including anxiety and headache. Caffeine is known to antagonize adenosine receptors in the brain. Adenosine causes sedation and relaxation when it acts upon its receptors. Caffeine prevents this action and thereby suppresses relaxation and sedation, and causes alertness and wakefulness. This inhibition of adenosine can influence the dopamine, serotonin, acetylcholine, and adrenaline systems.

Certain neurotransmitters are involved in motivation, attention, and learning. Examples of such neurotransmitters include dopamine, norepinephrine, and serotonin. Neurotransmitters may be recycled by re-uptake mechanisms, but there are many mechanisms at play. Monoamine oxidase is an enzyme in the brain that breaks down neurotransmitters while they are in the synaptic cleft, and thereby prevents re-uptake. This process is so significant that a class of neurological drugs, namely monoamine oxidase inhibitors (MAOIs), inhibit this pathway specifically to increase levels of neurotransmitters. The composition described herein provides resources to counter the effect or impact of this degradation, by including the building blocks for neurotransmitters. Such building blocks include precursors for neurotransmitters such as dopamine, norepinephrine, and serotonin. Thus by providing these precursors, neurotransmitters that have been depleted due to stress or cognitive load may be replenished. The amounts of such precursors in the composition are set to avoid adverse effects such as, for example, upset stomach or headache.

In the composition described herein, caffeine is paired with ingredients that reduce its undesirable properties and ingredients that are mood enhancers, meaning that they maintain a healthy mood balance specifically during prolonged cognitive exertion. The dosage amount of caffeine in the composition is between about 50 and about 250 mg. In certain embodiments, the dosage amount is 200 mg. The following ingredients are included in the composition together with caffeine: L-tyrosine, L-theanine, Rhodiola rosea extract, pyridoxine HCL, and 5-HTP. One or more other ingredient, such as a suitable carrier, excipient, vehicle, flavoring agent, stabilizer, etc., as generally known in the art, may also be included in the composition.

L-tyrosine, which is the amino acid precursor to several neurotransmitters, including dopamine and norepinephrine, is able to cross the blood-brain barrier, so it is able to enter the brain from an oral dosage. In contrast to the composition described herein, many products and formulations use an acetylated version of L-tyrosine, N-acetyl-L-tyrosine (NALT), often due to its improved solubility. Unlike L-tyrosine, however, NALT has been shown by numerous studies to be incapable of effectively crossing the blood-brain barrier, and to have no significant impact on cognitive processes (Magnusson, I., et al., (1989) Metabolism 38(10): 957-961). The dosage amount of L-tyrosine in the composition is between about 200 and about 1000 mg. In certain embodiments, the dosage amount is 500 mg.

L-theanine acts to reduce anxiety, diminish severity and frequency of headache, and attenuate increases in blood pressure that result from stress and/or caffeine consumption (Yoto, A. et al., (2012) J. Physiol. Anthropol. 31(1): 28). L-theanine is an amino acid that is not common in the human diet. L-theanine is structurally similar to L-glutamine and the neurotransmitters that are produced from it (e.g., GABA and glutamate). L-theanlne is known to reach the brain and act in the brain following an oral dosage. L-theanine induces relaxation without sedation, and is also implicated in reducing the perception of stress and slightly improving attention. L-theanine is useful in attenuating the “edge,” or overstimulation, of stimulants such as caffeine. A combination of L-theanine with caffeine has been shown through several studies in healthy humans to be synergistic in promoting cognition and attention (Kahathuduwa C. N., et al., (2016) Nutr. Neurosci. (On-line) 1-9; Haskell C. F., et al. (2008) Biol. Psychol. 77(2):113-22; Giesbrecht T. at al. (2010) Nutr Neurosci. 13(6):283-90; and Owen G. N. et al. (2008) Nutr. Neurosci. 11(4):193-8). The dosage amount of L-theanine in the composition is between about 50 and about 400 mg. In certain embodiments, the dosage amount is 160 mg.

Extracts obtained from the herb Rhodiole rosea have been shown to reduce effects of stress and fatigue and improve cognition in fatigued individuals and people with a high workload (Khanum, F., at al., (2005) Comprehensive Reviews in Food Science and Food Safety 4: 55-62). Rhodiola rosea extract has been shown to reliably reduce symptoms of stress in persons fatigued from non-exercise related stressors (Shevstov, V. A., et al., Phytomedicine 10 (2003): 95-105). Rhodiola rosea extracts are typically standardized for rosavin and/or salidroside content (Khanum, F., et al., (2005) Comprehensive Reviews in Food Science and Food Safety 4: 55-62). Research shows that rosavins have little therapeutic benefit, and that salidroside is the key agent responsible for the health benefits. The Rhodiola rosea extract used in the composition described herein is standardized to 3% salidroside. The dosage amount of Rhodiola rosea extract in the composition is between about 20 and about 200 mg. In certain embodiments, the dosage amount is 80 mg.

Pyridoxine HCL aids conversion of precursors to active neurotransmitters. Pyridoxine is a form of vitamin B6, which is converted by cells to pyridoxal phosphate (PLP). Other forms of vitamin B6 include pyridoxamine and pyridoxal. The requirement of pyridoxine in the central nervous system (CNS) is 100-fold greater than in the peripheral organs (Yarlagadda, A., et al., (2007) Psychiatry (Edgmont) 4(8): 58-60). In humans, an exogenous source of vitamin B6 is required for amino acid metabolism. PLP is critical for many cellular processes in the human body. Specifically, this molecule is used by enzymes to convert neurotransmitter precursors into the active neurotransmitter. For example, L-Dopa decarboxylase converts L-Dopa into dopamine, and this enzyme is dependent on PLP. The dosage amount of pyridoxine HCL in the composition is between about 10 and about 50 mg. In certain embodiments, the dosage amount is 40 mg.

5-HTP is a clinically-effective precursor to the neurotransmitter serotonin (Birdsall, T. C. (1998) Alternative Medicine Review 3(4): 271-280). 5-HTP acts to improve the mood of a subject by increasing serotonin synthesis in the brain. Serotonin is one of the principal neurotransmitters involved in healthy mood balance and feelings of joy. High dosages of 5-HTP are often used as a sleep aid, as serotonin is subsequently converted into melatonin, which is known to have sedative properties. Low dosages of 5-HTP are therefore included in the compositions described herein to minimize any potential sedating effects. The dosage amount of 5-HTP in the composition is between about 5 and about 50 mg. In certain embodiments, the dosage amount is 20 mg.

In one embodiment, the composition is consumed at any time regardless of stomach contents. In one embodiment, the composition is consumed on an empty stomach. The term “empty stomach” refers to a state in which the subject has an absence of protein in the subject's stomach. In one embodiment, this means no protein has been consumed for about 8 hours prior to ingestion of the composition described herein. In another embodiment, no protein has been consumed for about 2 hours prior to ingestion of the composition described herein. In another embodiment, no protein has been consumed for about 1 hour prior to ingestion of the composition described herein. In one embodiment, no protein is consumed for about one hour after consumption of a composition described herein. Consumption on an empty stomach is suggested because certain dietary amino acids could compete with uptake of the amino acids (e.g., L-tyrosine) in the composition (Wurtman, R. J., et al., (1981) Pharmacological Reviews 32(4): 315-335. Thus, efficacy of the composition may be maximized by consumption of the composition in the absence of other amino acids (i.e., on an empty stomach).

Exemplary dosage amounts for ingredients of the composition are presented in Table 1. For example, such dosage amounts may be provided in a single capsule or in two or more capsules. The dosages were developed in consideration of potential adverse effects (e.g., sedation) that have been reported at higher dosages of certain ingredients (e.g., 5-HTP and/or L-theanine), and also considering synergies between ingredients (such as caffeine and L-theanine) (Giesbrecht, T., et al., (2010) Nutritional Neuroscience 13(6)), 283-290.

The following working examples describe studies that have been conducted using a composition as described herein. Results include a decrease and/or elimination of negative side effects of caffeine for 18 subjects having a history of negative side effects after caffeine ingestion. Also, case studies were conducted on two subjects with neurological or psychological conditions. These individuals reported improvement in cognitive function after ingestion of the composition described herein.

In conclusion, compositions of active ingredients are described that acutely enhance and support cognitive function and diminish or eliminate negative side effects of caffeine ingestion. Although the working examples show that a composition according to the embodiments may help with certain conditions that are known to inhibit cognitive performance, they are also beneficial for individuals who do not have neurological or psychological conditions, but who merely wish to experience improved cognitive performance and/or decreased side effects of caffeine ingestion.

While the invention has been described with reference to the examples provided, it is to be understood that the invention is not limited by this description. To the contrary, the invention is intended to cover various modifications and equivalent arrangements included within the scope of the appended claims.

All cited publications are herein incorporated by reference in their entirety to the same extent as if each individual publication was specifically and individually indicated to be incorporated by reference in its entirety.

### WORKING EXAMPLES

### Example 1. Study with Subjects that are Prone to Side Effects from Caffeine

A study was conducted whereby subjects prone to negative side effects from caffeine were treated using the compositions and methods described herein. Specifically, 18 healthy male study participants (22-28 years old) with self-reported consistent experience of caffeine-induced negative side effects were studied. Each subject consistently experienced at least one of the following side effects following caffeine ingestion: headache, jitteriness, insomnia, and/or crash. Crash is characterized by feeling worse several hours after caffeine consumption compared to before consumption. The subjects were treated with an effective dosage of the composition described herein (see suggested dosage in Table 1). No other caffeine source such as a stimulant or dietary supplement was ingested within 24 hours of the study. The treatment was administered orally to participants on an empty stomach.

Improvements in cognitive performance (specifically, energy level, attention, and motivation) and subjective well-being were noted in all participants at the administered dosage. With the exception of sleeping difficulties in those subjects who previously reported a disposition to insomnia from caffeine, all negative side effects originally reported from traditional caffeine consumption were eliminated in the treatment group.

### Example 2. Case Study with Subject with Encephalopathy and ADHD Showing Cognition Performance Improvement

An improvement in cognitive performance in a female subject (34 years of age) with cognitive difficulties due to chronic static encephalopathy (resulting from cancer treatment) as well as diagnosed Attention Deficit Hyperactivity Disorder (ADHD). Subject was unable to continue prescription medication (i.e., methylphenidate) to enhance cognitive performance due to its negative side effects, which included heart palpitations. An effective dosage of the composition described herein (see suggested dosage in Table 1) was administered daily for eight days. Throughout this period, the subject noted a drastic improvement in subjective well-being, along with increased alertness and attention. No negative side effects were reported.

### Example 3. Case Study with Subject with Susceptibility to Negative Side Effects of Caffeine and Predominantly Inattentive Type Attention Deficit Hyperactivity

Disorder showing Cognition Performance Improvement Improved performance was seen for a female subject (24 years of age) with cognitive difficulties due to diagnosed Predominantly Inattentive Type Attention Deficit Hyperactivity Disorder (ADD). Subject reported susceptibility to negative side effects from consumption of stimulants, including caffeine-based stimulants. These side effects included anxiety and crash.

An effective dosage of the composition (see suggested dosage in Table 1) was administered on an as-needed basis on an empty stomach, with subjective improvement in alertness and attentiveness noted within the first hour of treatment. During treatment, the subject consistently noted improvement in attention and motivation to complete tasks, while reporting no negative side effects.

TABLE 1
An ingredient list for embodiments of the composition

| | | Suggested Amount per |
|---|---|---|
| Ingredient | Dosage Range (mg) | dosage (mg) |
| L-tyrosine | 200-1000 | 500 |
| caffeine | 50-250 | 200 |
| L-theanine | 50-400 | 160 |
| Rhodiola rosea extract | 20-200 | 80 |
| (at 3% salidroside) | | |
| Vitamin B6, specifically | 10-50 | 40 |
| pyridoxine HCL | | |
| Serotonin precursor, | 5-50 | 20 |
| specifically 5-HTP | | |

We claim:

1. A composition comprising L-tyrosine, caffeine, L-theanine, Rhodiola rosea extract, vitamin B6, and a serotonin precursor.

2. The composition of claim 1, wherein the vitamin B6 is pyridoxine, pyridoxamine, pyridoxal, or a salt thereof.

3. The composition of claim 1, wherein the serotonin precursor is 5-hydroxytryptophan (5-HTP).

4. The composition of claim 1, wherein a dosage amount of L-tyrosine is about 200 to about 1000 mg.

5. The composition of claim 1, wherein a dosage amount of caffeine is about 50 to about 250 mg.

6. The composition of claim 1, wherein a dosage amount of L-theanine is about 50 to about 400 mg.

7. The composition of claim 1, wherein a dosage amount of Rhodiola rosea extract is about 20 to about 200 mg.

8. The composition of claim 7, wherein the Rhodiola rosea extract comprises about 3% salidroside.

9. The composition of claim 1, wherein the dosage amount of pyridoxine HCl is about 10 to about 50 mg.

10. The composition of claim 3, wherein the dosage amount of 5-HTP is about 5 to about 50 mg.

11. The composition of claim 1, wherein the composition is orally-administered or transdermally-administered.

12. The composition of claim 11, wherein the composition is in capsule form, powder form, liquid form, or is in food or drink.

13. The composition of claim 1, comprising 500 mg of L-tyrosine, 200 mg of caffeine, 160 mg of L-theanine, 80 mg of Rhodiola rosea extract, 40 mg of pyridoxine hydrochloride, and 20 mg of 5-hydroxytryptophan (5-HTP).

14. A method of improving cognitive performance of a subject, comprising administering an effective amount of the composition of claim 1 to the subject.

15. The method of claim 14, wherein the subject has a neurological or psychological condition.

16. The method of claim 15, wherein the subject has Attention Deficit Hyperactivity Disorder (ADHD).

17. The method of claim 14, wherein the composition is ingested on an empty stomach.

18. A composition for enhancing cognitive function of a subject, comprising L-tyrosine, caffeine, L-theanine, Rhodiola rosea extract, vitamin B6, and a serotonin precursor.

19. The composition of claim 18, wherein the vitamin B6 is pyridoxine hydrochloride and the serotonin precursor is 5-hydroxytryptophan (5-HTP).

20. The composition of claim 18, wherein the composition comprises 500 mg of L-tyrosine, 200 mg of caffeine, 160 mg of L-theanine, 80 mg of Rhodiola rosea extract, 40 mg of pyridoxine hydrochloride, and 20 mg of 5-hydroxytryptophan (5-HTP).
